(19) United States
(12) Patent Application Publication
(10) Pub. No.: US 2012/0156681 A1
(43) Pub. Date: Jun. 21, 2012

(54) COMPOSITION FOR DIAGNOSIS OF LIVER METASTASIS OF COLORECTAL CANCER AND THE USE THEREOF

(76) Inventors: Woo Yong LEE, Seoul (KR); Yong Beom Cho, Seoul (KR); Ho-Kyung Chun, Seoul (KR)

(21) Appl. No.: 13/217,225

(22) Filed: Aug. 24, 2011

(30) Foreign Application Priority Data
Dec. 16, 2010 (KR) 10-2010-0129207

(51) Int. Cl. C12Q 1/68; G01N 33/574; C07K 16/24; C07H 21/02

(52) U.S. Cl. 435/6.12; 536/24.31; 536/24.5; 435/7.1; 530/389.2

(57) ABSTRACT

The present invention relates to a composition for diagnosis of liver metastasis of colorectal cancer and the use thereof, and more particularly to a composition for diagnosis of liver metastasis of colorectal cancer, which comprises either a substance for measuring the mRNA level of CCL7 (Chemokine (C-C motif) ligand 7) gene or a substance for measuring the level of a protein which is encoded by the gene. According to the present invention, whether liver metastasis of colorectal cancer occurred can be diagnosed by measuring the mRNA expression level of the CCL7 gene or the expression level of the CCL7 protein, and the use of the composition comprising an inhibitor of CCL7 gene allows the treatment of colorectal cancer or the liver metastasis of colorectal cancer.

### TECHNICAL FIELD

The present invention relates to a composition for diagnosis of liver metastasis of colorectal cancer and the use thereof, and more particularly to a composition for diagnosis of liver metastasis of colorectal cancer, which comprises a substance for measuring the mRNA level of CCL7 (Chemokine (C-C motif) ligand 7) gene or a substance for measuring the level of a protein which is encoded by the gene.

### BACKGROUND ART

Colorectal cancer is the third most common cancer in the world. In Korea, colorectal cancer is the fourth most common cancer after stomach, lung and liver cancers in men, and is the third most common cancer after breast and stomach cancers in women. Recently, as Korean eating habits have become westernized, the incidence of colorectal cancer has increased rapidly in Korea. During recent 10 years in Korea, the mortality caused by colorectal cancer has increased by about 80% and showed a tendency to increase gradually (Korean Central Cancer Registry 2002). About 50% patients with colorectal cancer die within 5 years after diagnosis, and 15-25% of the patients are found to have liver metastasis at the time of diagnosis, and 20-30% of the patients are found to have liver metastasis during follow-up observation after surgery of primary colorectal cancer. Indeed, the cause of death of patients with malignant tumors, including colorectal cancer, is due to the metastasis of malignant tumors rather than the malignant tumors themselves.

Cancer-related studies have mostly been conducted on carcinogenic processes and mechanisms. In order to improve the treatment and survival rate of colorectal cancer patients, studies on colorectal cancer metastasis which is the most common cause of death are required, and among them, studies on the metastasis of colorectal cancer to the liver that is the most common metastasis site are necessary.

In many studies conducted to date, a microarray gene expression profiling technique has been used to discover biomarkers related to the occurrence or metastasis of malignant tumors. Although this technique allows the expression of whole genes to be determined, it is difficult to carry out, is very expensive and has high false-positivity. Also, additional processes of examining the expression levels of genes by Northern blotting, RT-PCR and the like should be performed after the test. The RT² Profiler PCR Array comprises a 96-well plate containing SYBR Green-optimized primer sets for genes classified according to pathway (e.g., apoptosis, cell cycle, cancer, signal pathway, etc.) or disease and can analyze the expression of a group of groups involved in a specific signal pathway using a real time PCR-based technique.

Accordingly, the present inventors have made extensive efforts to develop a biomarker capable of diagnosing the liver metastasis of colorectal cancer and, as a result, have found that, when the mRNA expression level of CCL7 gene and the level of CCL7 protein are compared between the liver metastasis site of colorectal cancer and the primary site of colorectal cancer, the mRNA level of the CCL7 gene is more strongly expressed at the liver metastasis site and the level of the CCL7 protein is also higher at the liver metastasis site, suggesting that the CCL7 gene can be used as a biomarker specific for liver metastasis of colorectal cancer, thereby completing the present invention.

### DISCLOSURE OF INVENTION

### Technical Problem

It is an object of the present invention to provide a composition for diagnosis of liver metastasis of colorectal cancer, which comprises either a substance for measuring the mRNA level of CCL7 (Chemokine (C-C motif) ligand 7) gene or a substance for measuring the level of a protein which is encoded by the gene, and a kit for diagnosis of liver metastasis of colorectal cancer, which comprises the composition.

Another object of the present invention is to provide a composition for inhibiting liver metastasis of colorectal cancer, which comprises an inhibitor of CCL7 (Chemokine (C-C motif) ligand 7) gene.

Yet another object of the present invention is to provide a method of providing information for diagnosis of liver metastasis of colorectal cancer by measuring the mRNA expression level of CCL7 (Chemokine (C-C motif) ligand 7) gene or the expression level of CCL7 protein.

### Technical Solution

To achieve the above objects, the present invention provides a composition for diagnosis of liver metastasis of colorectal cancer, which comprises a substance for measuring the mRNA level of CCL7 (Chemokine (C-C motif) ligand 7) gene or a substance for measuring the level of a protein which is encoded by the gene.

The present invention also provides a kit for diagnosis of liver metastasis of colorectal cancer, which comprises said composition for diagnosis of liver metastasis of colorectal cancer.

The present invention also provides a composition for inhibiting liver metastasis of colorectal cancer, which comprises an inhibitor of CCL7 (Chemokine (C-C motif) ligand 7) gene.

The present invention also provides a method for providing information for diagnosis of liver metastasis of colorectal cancer, the method comprising the steps of: (a) measuring the mRNA level of CCL7 gene in a biological sample isolated from a patient suspected to have liver metastasis of colorectal cancer; and (b) comparing the measured mRNA level of CCL7 gene with the mRNA level of CCL7 gene in a control sample obtained from primary colorectal cancer tissue.

The present invention also provides a method for providing information for diagnosis of liver metastasis of colorectal cancer, the method comprising the steps of: (a) measuring the level of CCL7 protein in a biological sample isolated from a patient suspected to have liver metastasis of colorectal cancer; and (b) comparing the measured protein level with the level of CCL7 protein in a control sample obtained from primary colorectal cancer tissue.

According to the present invention, whether liver metastasis of colorectal cancer occurred or not can be diagnosed by measuring the mRNA level of CCL7 gene or the expression level of CCL7 protein. In addition, the use of the composition comprising the inhibitor against CCL7 gene allows the treatment of colorectal cancer or the inhibition of liver metastasis of colorectal cancer.

### BRIEF DESCRIPTION OF THE DRAWINGS

FIG. 1 shows the results of analysis of target genes showing a significant difference in gene expression between primary colorectal cancer and liver-metastasized colorectal cancer.

FIG. 2(A) shows a comparison of the expression of CCL7 between the primary site of colorectal cancer and the liver metastasis tissue of colorectal cancer, and FIG. 2(B) shows a comparison of the expression of the CCL7 receptors CCR1, CCR2 and CCR3 between the primary site of colorectal cancer and the liver metastasis tissue of colorectal cancer.

FIG. 3(A) is a photograph of immonohistochemical staining of CCL7 in normal colorectal tissue (H&E, ×200). FIG. 3(B) is a photograph of immonohistochemical staining of CCL7 in primary colorectal cancer tissue (H&E, ×200). FIG. 3(C) is a photograph of immonohistochemical staining of CCL7 in the liver metastasis tissue of colorectal cancer (H&E, ×200).

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein is one which is well known and commonly employed in the art.

The present inventors used an RT² Profiler PCR array to discover a biomarker associated with liver metastasis of colorectal cancer. The Profiler PCR array comprises a 96-well plate containing SYBR Green-optimized primer sets for genes, classified according to pathway (e.g., apoptosis, cell cycle, cancer, signal pathway, etc.) or disease, and allows the expressions of genes in a specific signal pathway to be analyzed at the same time using a real-time PCR-based technique.

CCL7 (Chemokine (C-C motif) ligand 7) discovered as a marker of liver metastasis of colorectal cancer in the present invention is a small chemokine which was previously called “monocyte-specific chemokine 3” (MCP-3). CCL7 has two adjacent N-terminal cysteine residues and serves to attract monocytes and to control the function of macrophages. Such CCL7 is known to be secreted from monocytes, fibroblasts, platelets, colonic epithelial cells and some malignant tumor cells and acts as a lymphocyte chemoattractant by binding to three chemokine receptors (CCR1, CCR2, and CCR3).

In one aspect, the present invention is directed to a composition for diagnosis of liver metastasis of colorectal cancer, which comprises a substance for measuring the mRNA level of CCL7 (Chemokine (C-C motif) ligand 7) gene or a substance for measuring the level of a protein which is encoded by the gene.

In the present invention, the substance for measuring the mRNA level of CCL7 gene is preferably a probe having a complementary sequence, specific for the CCL7 gene, and the use of the probe specific for the gene allows to be effectively detected at the mRNA level.

As used herein, the term “probe” means a nucleic acid fragment such as RNA or DNA, which can specifically bind to mRNA and has a length of several bases to several hundred bases. The probe is labeled so that the presence or absence of a specific mRNA can be determined. The probe can be constructed in the form of an oligonucleotide probe, a single stranded DNA probe, a double stranded DNA probe, an RNA probe or the like.

In the present invention, the substance capable of measuring the expression level of the protein which is encoded by the CCL7 gene may be an antibody or a fragment thereof, which binds specifically to the CCL7 protein. Herein, the antibody is intended to include all polyclonal antibodies, monoclonal antibodies and recombinant antibodies and means a specific molecular molecule directed toward antigenic sites.

A polyclonal antibody which is the liver disease marker protein can be produced by a method well known in the art, which includes injecting TM4SF5 antigen into an animal and collecting blood from the animal to obtain serum containing antibodies. This polyclonal antibody can be prepared from any animal host, such as goats, rabbits, sheep, monkeys, horses, pigs, cows and dogs. A monoclonal antibody can be prepared by a method well known in the art, such as a hybridoma method (see Kohler and Milstein (1976) European Journal of Immunology 6:511-519) or a phage antibody library technique (Clackson et al, Nature, 352:624-628, 1991; Marks et al, J. Mol. Biol., 222:58, 1-597, 1991). The antibody prepared by the above methods may be isolated using gel electrophoresis, dialysis, salting out, ion exchange chromatography, affinity chromatography, etc. In addition, the antibody of the present invention includes functional fragments of antibody molecules, as well as a complete form having two full-length light chains and two full-length heavy chains. The functional fragment of antibody molecules means a fragment having at least an antigen-binding function, and examples thereof. Fab, F(ab′)2, Fv, and the like.

In another aspect, the present invention is also directed to a kit for diagnosis of liver metastasis of colorectal cancer, which comprises said composition for diagnosis of liver metastasis of colorectal cancer.

The diagnostic kit of the present invention is composed of one or more compositions, solutions or instruments, which are suitable for analysis methods. It may be a RT-PCR kit, a DNA chip kit or a protein chip kit. The RT-PCR kit may comprise, in addition to each primer pair specific for the marker gene, a test tube or another suitable container, a reaction buffer, deoxynucleotides (dNTPs), enzymes such as Taq-polymerase, reverse transcriptase and DNase, an RNase inhibitor, DEPC-water, sterile water, etc. Also, it may comprise a primer pair specific for a gene which is used as a quantitative control. The DNA chip kit may comprise a substrate, a gene or corresponding cDNA attached as a probe to the substrate, and optionally a quantification control gene or corresponding cDNA attached to the substrate.

In addition, the diagnostic kit according to the present invention may comprise an agent for measuring the level of CCL7, in which the agent for measuring the protein is preferably an antibody specific for the protein. Thus, the diagnostic kit comprising the agent for measuring the protein level may be a kit for detection of diagnostic markers, which comprises essential elements required for carrying out, for example, ELISA. This kit may also comprise reagents that may detect bound antibodies, for example, labeled secondary antibodies, chromophores, enzymes (e.g., conjugated with antibodies) and their substrates. Also, it may comprise an antibody specific for a control protein for quantification.

Further, the amount of antigen-antibody complexes formed may be quantitatively determined by measuring the signal intensity of a detection label. Such a detection label may be selected from the group consisting of, but not limited to, enzymes, fluorescent substances, ligands, luminescent substances, microparticles, redox molecules and radioactive isotopes. Methods for measuring protein levels include, but not limited to, Western blotting, ELISA (enzyme linked immunosorbent assay), RIA (Radioimmunoassay), radioimmunodiffusion, Ouchterlony immunodiffusion, rocket immunoelectrophoresis, immunohistostaining, immunoprecipitation, complement fixation assay, FACS and protein chip assays.

In still another aspect, the present invention is also directed to a composition for inhibiting liver metastasis of colorectal cancer, which comprises an inhibitor of CCL7 (Chemokine (C-C motif) ligand 7) gene.

The inhibitor against the CCL7 (Chemokine (C-C motif) ligand 7) gene may be an antisense oligonucleotide against the mRNA of the CCL7 gene, or it may be a siRNA of the CCL7 gene.

As used herein, the term “siRNA” (small interfering RNA) means a short double-strand RNA (dsRNA) that mediates efficient gene silencing in a sequence-specific manner. SiRNAs has the potential to be a very potent drug for the inhibition of specific gene expression in vivo in light of its long-lasting effectiveness in cell cultures and in vivo, its ability to transfect cells in vivo, and its resistance to degradation in serum. siRNA can exhibit effects equal or higher than those of antisense oligonucleotides even at relatively low concentrations, and thus has been proposed as an alternative for antisense oligonucleotides. A person skilled in the art can synthesize and modify the antisense oligonucleotide and siRNA in a desired manner using a method known in the art.

In yet another aspect, the present invention is also directed to a method for providing information for diagnosis of liver metastasis of colorectal cancer, the method comprising the steps of: (a) measuring the mRNA level of CCL7 gene in a biological sample isolated from a patient suspected to have liver metastasis of colorectal cancer; and (b) comparing the measured mRNA level of CCL7 gene with the mRNA level of CCL7 gene in a control sample obtained from primary colorectal cancer tissue.

In a further aspect, the present invention is also directed to a method for providing information for diagnosis of liver metastasis of colorectal cancer, the method comprising the steps of: (a) measuring the level of CCL7 protein in a biological sample isolated from a patient suspected to have liver metastasis of colorectal cancer; and (b) comparing the measured level of CCL7 protein with the level of CCL7 protein in a control sample obtained from primary colorectal cancer tissue.

In the present invention, the step of measuring the level of CCL7 protein in the biological sample isolated from the patient suspected to have liver metastasis of colorectal cancer can be performed by brining the inventive composition for diagnosis of liver metastasis of colorectal cancer into contact with the biological sample. Also, the step of comparing the measured protein level with the level of CCL7 protein in a control sample obtained from primary colorectal tissue includes determining whether the level of CCL7 protein in the sample is higher that of CCL7 in the control sample, thereby providing information for diagnosis of liver metastasis of colorectal cancer.

According to such methods of the present invention, by comparing the expression level of CCL7 protein in the primary colorectal cancer control sample with the expression level of CCL7 protein in the sample isolated from the patient suspected to have liver metastasis of colorectal cancer, a patient having liver metastasis of colorectal cancer can be diagnosed and, furthermore, the progression or prognosis of colorectal cancer can be predicted.

In the present invention, the step of measuring the level of CCL7 protein can be performed by measuring the level of CCL7 protein using an antibody that specifically recognizes CCL7. Specifically, the step can be performed using the above-mentioned various methods of measuring protein levels, including Western blotting, ELISA (enzyme linked immunosorbent assay), RIA (Radioimmunoassay), radioimmunodiffusion, Ouchterlony immunodiffusion, rocket immunoelectrophoresis, immunohistostaining, immunoprecipitation, complement fixation assay, FACS, and protein chip assays.

In the present invention, the biological sample may be tissue, cell, whole blood, serum or plasma.

Hereinafter, the present invention will be described in further detail with reference to examples. It will be obvious to those skilled in the art that these examples are illustrative purposes only and are not to be construed to limit the scope of the present invention.

### EXAMPLES

### Example 1

### Discovery of Gene Specific for Liver Metastasis of Colorectal Cancer

To analyze target genes showing a significant difference in gene expression between primary colorectal cancer and liver-metastasized colorectal cancer, RNA was extracted from the colorectal cancer tissue and liver-metastasized colorectal cancer tissue (fresh frozen tissue) of six patient (Samsung Medical Center, Korea), who had liver metastasis of colorectal cancer and underwent surgery (colorectal and liver resection). The extracted RNA was subjected to RT² Profiler PCR Array analysis.

Specifically, the colorectal cancer tissue and liver-metastasized colorectal cancer tissue of the six patients were frozen at −80° C. until use. The frozen tissues were stained with H&E, and necrotic tumor tissues and intervening normal tissues were removed therefrom. Then, the total RNA was extracted from the frozen tissues using a Nucleospin RNA kit. cDNA was synthesized from the extracted RNA using a RT² First Strand Synthesis Kit (Super Array Bioscience, Frederick, Md.) and analyzed using a Human tumor metastasis PCR array and a RT² SYBR Green/Rox PCR mastermix [APMM012C and PA-012-24, (Super Array Bioscience, Frederick, Md.)].

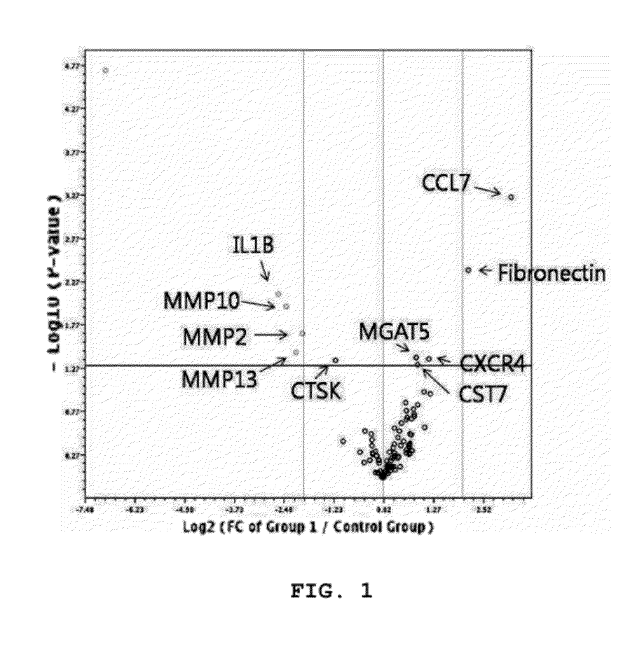

As a result, it was found that the expressions of CCL7 (p=0.0006), FN1 (p=0.0039), CXCR4 (p=0.0420), CST7 (p=0.0491) and MGAT5 (p=0.0407) were higher in the liver-metastasized colorectal cancer tissue than in the primary colorectal cancer tissue, and among them CCL7 (p=0.0006) showed the highest expression level (see FIG. 1).

### Example 2

### 2-1: mRNA Expression Level of CCL7 Gene in Liver-Metastasized Colorectal Cancer Tissue

In order to examine whether the CCL7 gene which showed an expression level higher in the liver-metastasized colorectal cancer tissue than in the primary colorectal cancer tissue as described in Example 1 functicns as a marker of liver metastasis of colorectal cancer, the following experiment was carried out.

Total RNA was extracted from paraffin blocks using the MasterPure™ Complete DNA and RNA Rurification Kit (Epicentre Biotechnologies). mRNA was amplified and then transcribed from double-stranded cDNA using SuperScript™ III Reverse transciptase (Invitrogen). Quantitative real-time RT PCR of the mRNA was performed three times in a 384-well plate. Each PCR reaction was performed 5 μL of Power SYBR®Green PCR Master Mix (Applied Biosystems, Inc., Foster City, Calif.), 0.25 μL of 10 μM primer, and a probe set of CCL7 (Bioneer Oligo Synthesis Report), CCR1 (Bioneer Oligo Synthesis Report), CCR2 (Bioneer Oligo Synthesis Report), CCR3 (Bioneer Oligo Synthesis Report) and GAPDH (Bioneer Oligo Synthesis Report). The primer sets used in the PCR reactions are shown in Table 1 below.

TABLE 1

| | | | SEQ |
|---|---|---|---|
| marker | | sequence | ID NO |
| CCL7 | sense | 5′-TGCTCAGCCAGTTGGGATTA-3′ | 1 |
| | antisense | 5′-GGACAGTGGCTACTGGTGGT′3′ | 2 |
| CCR1 | sense | 5′-CTGGTTGGAAACATCCTGGT-3′ | 3 |
| | antisense | 5′-GGAAGCGTGAACAGGAAGAG-3′ | 4 |
| CCR2 | sense | 5′-CCCCAGTCACCTGCTGTTAT-3′ | 5 |
| | antisense | 5′-GCTTCTTTGGGACACTTGCT-3′ | 6 |
| CCR3 | sense | 5′-GTGTTCACTGTGGGCCTCTT-3′ | 7 |
| | antisense | 5′-GTGACGAGGAAGAGCAGGTC-3′ | 8 |
| GAPDH | sense | 5′-ACCGTCAAGGCTGAGAA-3′ | 9 |
| | antisense | 5′-CATCGCCCCACTTGATT-3′ | 10 |

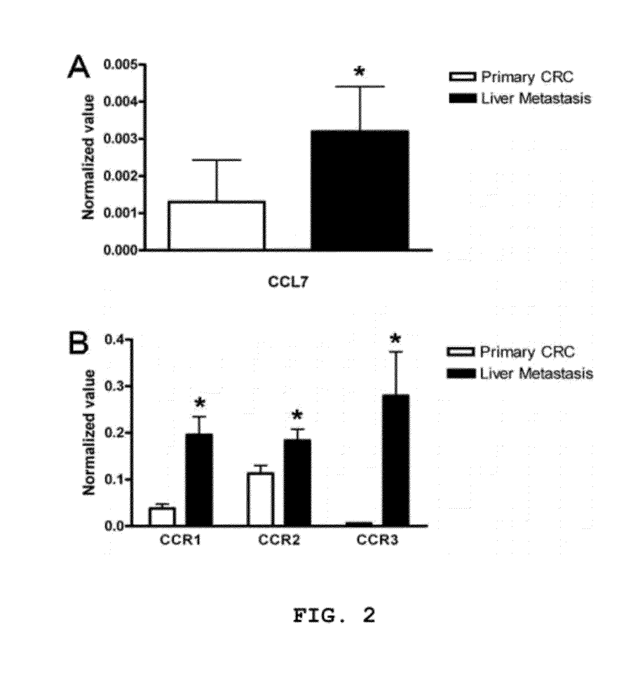

RT² Profiler PCR Array and real time PCR were performed on the primary colorectal cancer tissue and the liver-metastasized colorectal cancer tissue. As a result, the RNA expression of CCL7 was higher in the liver-metastasized colorectal cancer tissue than in the primary colorectal cancer tissue (FIG. 2A). Also, CCR1, CCR2 and CCR3 known as CCL7 receptors also showed RNA expression levels higher in the liver-metastasized colorectal cancer tissue than in the primary colorectal cancer tissue (see FIG. 2B).

### 2-2: Expression Level of CCL7 Protein in Liver-Metastasized Colorectal Cancer Tissue

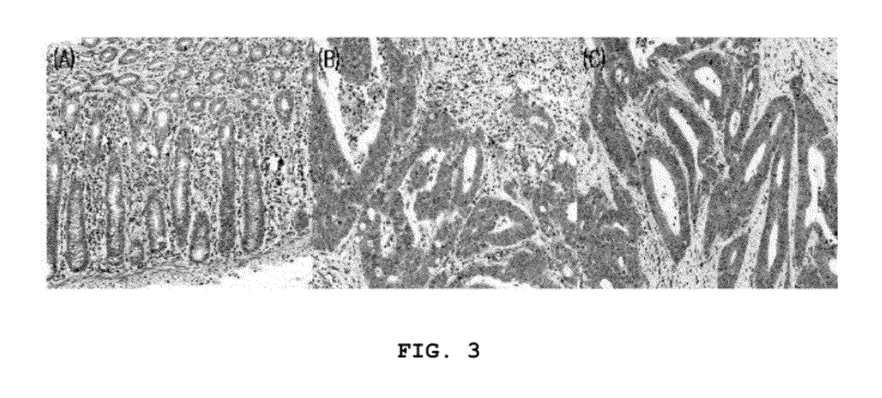

Immunohistochemical staining was performed using an antibody (rabbit anti-human polyclonal CCL7 (dilution 1:1000, GenWay Biotech, Inc., USA) against CCL7. As a result, although CCL7 was also expressed in normal colorectal tissue, the colorectal cancer tissue (see FIG. 3B) showed a strong expression of CCL7 as compared to the normal colorectal tissue (see FIG. 3A), and the liver-metastasized colorectal cancer tissue (see FIG. 3C) showed a strong expression of CCL7 as compared to the primary colorectal cancer tissue (see FIG. 3). It could be seen from such results that CCL7 is highly valuable as a marker specific for liver metastasis of colorectal cancer. If CCL7 acts as a marker specific for liver metastasis of colorectal cancer, an antibody targeting CCL7 can be developed and used as a targeted agent.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A composition for diagnosis of liver metastasis of colorectal cancer comprising a substance for measuring the mRNA level of CCL7 (Chemokine (C-C motif) ligand 7) gene or a substance for measuring the level of a protein which is encoded by the gene.

2. The composition of claim 1, wherein the substance for measuring the mRNA level of CCL7 is a probe having a complementary sequence specific for the CCL7.

3. The composition of claim 1, wherein the substance for measuring the level of CCL7 protein is an antibody or a fragment thereof that binds specifically to the protein which is encoded by the gene.

4. A kit for diagnosis of liver metastasis of colorectal cancer comprising a composition according to claims 1.

5. A composition for inhibiting liver metastasis of colorectal cancer comprising an inhibitor of CCL7 (Chemokine (C-C motif) ligand 7) gene.

6. The composition of claim 5, wherein the inhibitor against the CCL7 gene is an antisense oligonucleotide against the mRNA of the CCL7 gene.

7. The composition of claim 5, wherein the inhibitor against the CCL7 gene is a siRNA of the CCL7 gene.

8. A method for providing information for diagnosis of liver metastasis of colorectal cancer comprising the steps of:
(a) measuring the mRNA level of CCL7 gene in a biological sample isolated from a patient suspected to have liver metastasis of colorectal cancer; and
(b) comparing the measured mRNA level of CCL7 gene with the mRNA level of CCL7 gene in a control sample obtained from primary colorectal cancer tissue.

9. A method for providing information for diagnosis of liver metastasis of colorectal cancer comprising the steps of:
(a) measuring the level of CCL7 protein in a biological sample isolated from a patient suspected to have liver metastasis of colorectal cancer; and
(b) comparing the measured protein level of with the level of CCL7 protein in a control sample obtained from primary colorectal cancer tissue.

10. A kit for diagnosis of liver metastasis of colorectal cancer comprising a composition according to claims 2.

11. A kit for diagnosis of liver metastasis of colorectal cancer comprising a composition according to claims 3.


SEQUENCE LISTING (10 sequences)

SEQ ID NO: 1 - DNA, 20 bp, Artificial
  primer
tgctcagccagttgggatta

SEQ ID NO: 2 - DNA, 20 bp, Artificial
  primer
ggacagtggctactggtggt

SEQ ID NO: 3 - DNA, 20 bp, Artificial
  primer
ctggttggaaacatcctggt

SEQ ID NO: 4 - DNA, 20 bp, Artificial
  primer
ggaagcgtgaacaggaagag

SEQ ID NO: 5 - DNA, 20 bp, Artificial
  primer
ccccagtcacctgctgttat

SEQ ID NO: 6 - DNA, 20 bp, Artificial
  primer
gcttctttgggacacttgct

SEQ ID NO: 7 - DNA, 20 bp, Artificial
  primer
gtgttcactgtgggcctctt

SEQ ID NO: 8 - DNA, 20 bp, Artificial
  primer
gtgacgaggaagagcaggtc

SEQ ID NO: 9 - DNA, 17 bp, Artificial
  primer
accgtcaaggctgagaa

SEQ ID NO: 10 - DNA, 17 bp, Artificial
  primer
catcgccccacttgatt